(19) United States
(12) Patent Application Publication
(10) Pub. No.: US 2019/0152918 A1
(43) Pub. Date: May 23, 2019

(54) CRYSTALLINE CALCIUM SALT OF (S)-2-(DIPHENYLACETYL)-1,2,3,4-TETRAHYDRO-6-METHOXY-5-(PHENYLMETHOXY)-3-ISOQUINOLINE CARBOXYLIC ACID

(71) Applicant: RATIOPHARM GMBH, Ulm (DE)

(72) Inventor: Jens Geier, Hayingen (DE)

(21) Appl. No.: 16/313,703

(22) PCT Filed: Jun. 27, 2017
(86) PCT No.: PCT/EP2017/065771

§ 371 Date: Dec. 27, 2018

(30) Foreign Application Priority Data
Jun. 27, 2016 (EP) 16020252.9

(51) Int. Cl. C07D 217/26; A61P 25/00

(52) U.S. Cl. CPC C07D 217/26; C07B 2200/13; A61P 25/00

(57) ABSTRACT

The present invention relates to calcium salts of (S)-2-(diphenylacetyl)-1,2,3,4-tetrahydro-6-methoxy-5-(phenylmethoxy)-3-isoquinoline carboxylic acid in crystalline form and to a process for producing calcium salts of (S)-2-(diphenylacetyl)-1,2,3,4-tetrahydro-6-methoxy-5-(phenylmethoxy)-3-isoquinoline carboxylic acid in crystalline form. Further, the present invention relates to a composition comprising a crystalline calcium salt of (S)-2-(diphenylacetyl)-1,2,3,4-tetrahydro-6-methoxy-5-(phenylmethoxy)-3-isoquinoline carboxylic acid and to a dosage form containing the composition of the invention.

### BACKGROUND OF THE INVENTION

The present invention relates to calcium salts of (S)-2-(diphenylacetyl)-1,2,3,4-tetrahydro-6-methoxy-5-(phenylmethoxy)-3-isoquinoline carboxylic acid in crystalline form and to a process for producing calcium salts of (S)-2-(diphenylacetyl)-1,2,3,4-tetrahydro-6-methoxy-5-(phenylmethoxy)-3-isoquinoline carboxylic acid in crystalline form. Further, the present invention relates to a composition comprising a crystalline calcium salt of (S)-2-(diphenylacetyl)-1,2,3,4-tetrahydro-6-methoxy-5-(phenylmethoxy)-3-isoquinoline carboxylic acid and to a dosage form containing the composition of the invention.

(S)-2-(diphenylacetyl)-1,2,3,4-tetrahydro-6-methoxy-5-(phenylmethoxy)-3-isoquinoline carboxylic acid is represented by the following chemical structure according to Formula (I):

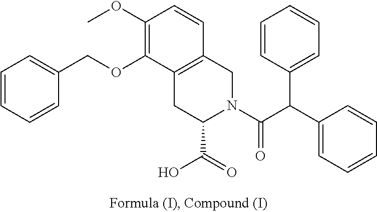 [SMILES: COc1ccc2c(c1OCc1ccccc1)C[C@@H](C(=O)O)N(C(=O)C(c1ccccc1)c1ccccc1)C2]

(S)-2-(diphenylacetyl)-1,2,3,4-tetrahydro-6-methoxy-5-(phenylmethoxy)-3-isoquinoline carboxylic acid is an analgesic, in particular for treating inflammatory pain and neuropathic pain, whose effect is reported to be based on AT₂ receptor antagonism.

Synthesis pathways for Compound I and its use as an analgesic are described in WO2006/066361 and WO2007/06938.

Further, in WO2012/010843 (WO'843), Compound I is reported to be present in form of salts, in particular as a sodium salt.

WO'843 relates to salts and solvates of compound I and demonstrates that the sodium salt is the only salt from the number of salt-forming agents studied, which leads to non-deliquescent solids. In particular, the sodium mono-ethanol solvate and the sodium hydrate seem to be disclosed. In Example 9 of WO'843 it is reported that when divalent salts, such as the calcium salt or the magnesium salt of Compound I were prepared in analogy to the sodium salt, only amorphous forms of the free acid of Compound I were obtained.

WO'843 substantially relates to the preparation of sodium mono-ethanol solvate and the sodium hydrate of compound I.

Discovering new salts, in particular divalent salts compared to monovalent salts, such as sodium salts, new solid state forms and new solvates of a pharmaceutical product can provide materials having desirable processing properties, such as ease of handling, ease of processing, storage stability, and ease of purification or as desirable intermediate crystal forms that facilitate conversion to other salts or polymorphic forms. New salts, polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life. For at least these reasons, there is a need for additional salts and solid state forms (including solvated forms) of (S)-2-(diphenylacetyl)-1,2,3,4-tetrahydro-6-methoxy-5-(phenylmethoxy)-3-isoquinoline carboxylic acid, for example of calcium (S)-2-(diphenylacetyl)-1,2,3,4-tetrahydro-6-methoxy-5-(phenylmethoxy)-3-isoquinoline carboxylate, i.e. a calcium salt of (S)-2-(diphenylacetyl)-1,2,3,4-tetrahydro-6-methoxy-5-(phenylmethoxy)-3-isoquinoline carboxylic acid (calcium salt of Compound I).

It was an object of the present invention to prepare new salts, in particular divalent salts compared to monovalent salts (such as sodium salts), new solid state forms and new solvates the of the compound I. It has been surprisingly found that contrary to the report in WO'843 crystalline calcium salts of Compound I can be obtained in a simple, economic way with excellent purity.

### SUMMARY OF THE INVENTION

The present disclosure relates to solid state forms of Compound I, in particular divalent salts thereof, to processes for preparation thereof, and to pharmaceutical compositions comprising these solid state forms.

The present disclosure also relates to the solid state forms of Compound I of the present disclosure, in particular divalent salts thereof, for use in the preparation of pharmaceutical compositions for use in medicine, preferably for the treatment of pain.

The present disclosure further encompasses processes to prepare said pharmaceutical composition of Compound I comprising combining a state form of Compound I and at least one pharmaceutically acceptable excipient.

### BRIEF DESCRIPTION OF THE DRAWINGS

FIG. 1 shows an X-ray powder diffraction (XRPD) pattern of a crystalline hydrate of the calcium salt of Compound I.

FIG. 2 shows an XRPD pattern of a crystalline ethanolate of the calcium salt of Compound I.

FIG. 3 shows a TGA thermogram of a crystalline hydrate of the calcium salt of Compound I.

FIG. 4 shows a TGA thermogram of a crystalline ethanolate of the calcium salt of Compound I.

### DETAILED DESCRIPTION OF ILLUSTRATIVE EMBODIMENTS

According to the present invention, the above objective can be achieved by providing crystalline calcium salts of Compound I, preferably as crystalline ethanol solvate of the calcium salt of Compound I and/or preferably as crystalline hydrate of the calcium salt of compound I, which can advantageously be further processed in a pharmaceutical composition or a dosage form.

Thus, a subject of the invention is a crystalline ethanol-solvate and a crystalline hydrate of the calcium salt of (S)-2-(diphenylacetyl)-1,2,3,4-tetrahydro-6-methoxy-5-(phenylmethoxy)-3-isoquinoline carboxylic acid.

A crystal form may be referred to herein as being characterized by data selected from two or more different data groupings, for example by a powder XRD pattern (XRPD), having a group of specific peaks or by a powder XRD pattern as depicted in a diffractogram, or by “a combination thereof” (or “combinations thereof” or “any combination thereof”). These expressions, e.g. “any combination thereof”, contemplate that the skilled person may characterize a crystal form using any combination of the recited characteristic analytical data. For example, the skilled person may characterize a crystal form using a group of three, four or five characteristic powder XRD peaks and supplement this characterization with one or more additional feature(s) observed in the powder X-ray diffractogram, e.g. an additional peak, a characteristic peak shape, a peak intensity or even the absence of a peak at some position in the powder XRD pattern. Alternatively, the skilled person may in some instances characterize a crystal form using a group of three, four or five characteristic powder XRD peaks.

In the present application, the XRPD is measured as described below in the experimental section. Further, unless indicated otherwise, XRPD peaks are reported as degrees 2θ values with a standard error of ±0.2 degrees 2θ.

A crystal form may be referred to herein as being characterized by graphical data “as depicted in” a particular figure. Such data include for example powder X-ray diffractograms. The skilled person will understand that such graphical representation of data may be subject to small variations, e.g. in peak relative intensities and peak positions, due to factors such as variations in instrument response and variations in sample preparation, which are well-known to the skilled person. Nonetheless, the skilled person would readily be capable of comparing the graphical data in the figures herein with graphical data generated for an unknown crystal form, and confirm as to whether the two sets of graphical data characterize the same crystal form or two different crystal forms.

The term “solvate”, as used herein and unless indicated otherwise, refers to a crystal form that incorporates a solvent in the crystal structure. When the solvent is water, the solvate is often referred to as a “hydrate”. The solvent in a solvate may be present in either a stoichiometric or in a non-stoichiometric amount. A “mono solvate” or “monohydrate”, as used herein and unless indicated otherwise, refers to one molecule of solvent or water per one (S)-2-(diphenylacetyl)-1,2,3,4-tetrahydro-6-methoxy-5-(phenylmethoxy)-3-isoquinoline carboxylate ion.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

In a preferred embodiment crystalline hydrate of the calcium salt of compound I can preferably have characteristic X-ray powder diffraction peaks at 5.6, 6.0, 11.9 and 16.8 degrees 2θ (±0.2 degrees 2θ).

In a further preferred embodiment the crystalline hydrate of the calcium salt of compound I can be further characterized by one or more further XRPD diffraction peak(s) at 21.2 and/or 23.3 degrees 2θ (±0.2 degrees 2θ), preferably at 21.2 degrees 2θ (±0.2 degrees 2θ).

In a preferred embodiment crystalline hydrate of the calcium salt of compound I can be characterized by one or more further XRPD diffraction peak(s) at 9.9, 15.0, 15.4 and/or 16.1 degrees 2θ (±0.2 degrees 2θ).

In an alternatively further preferred embodiment of the present invention crystalline hydrate of the calcium salt of compound I can be characterized by the XRPD diffraction peak(s) at degrees 2θ±0.2 degrees 2θ (intensity %): 5.6 (100), 6.0 (6.1), 9.9 (1.6), 10.5 (0.8), 11.2 (0.2), 11.9 (9.5), 12.9 (0.3), 13.3 (0.7), 14.5 (0.4), 15.0 (2.2), 15.4 (2.0), 16.1 (3.4), 16.7 (9.2), 16.8 (13.1), 17.3 (0.5), 17.9 (9.3), 18.2 (2.1), 18.6 (0.5), 19.3 (4.2), 19.5 (4.1), 19.9 (2.1), 20.3 (0.8), 21.2 (16.4), 21.5 (6.5), 22.2 (2.1), 22.5 (2.3) and 23.3 (8.3).

In an alternatively further preferred embodiment of the present invention crystalline hydrate of the calcium salt of compound I can be characterized by the XRPD diffraction peak(s) at degrees 2θ±0.2 degrees 2θ: 5.6, 6.0, 9.9, 10.5, 11.2, 11.9, 12.9, 13.3, 14.5, 15.0, 15.4, 16.1, 16.7, 16.8, 17.3, 17.9, 18.2, 18.6, 19.3, 19.5, 19.9, 20.3, 21.2, 21.5, 22.2, 22.5 and 23.3.

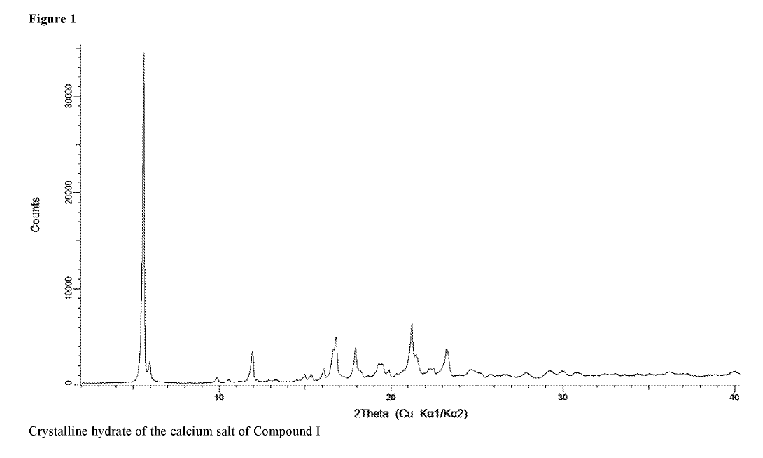

An XRPD diffraction pattern of a preferred embodiment of the crystalline hydrate of the calcium salt of compound I of the present invention is shown in FIG. 1.

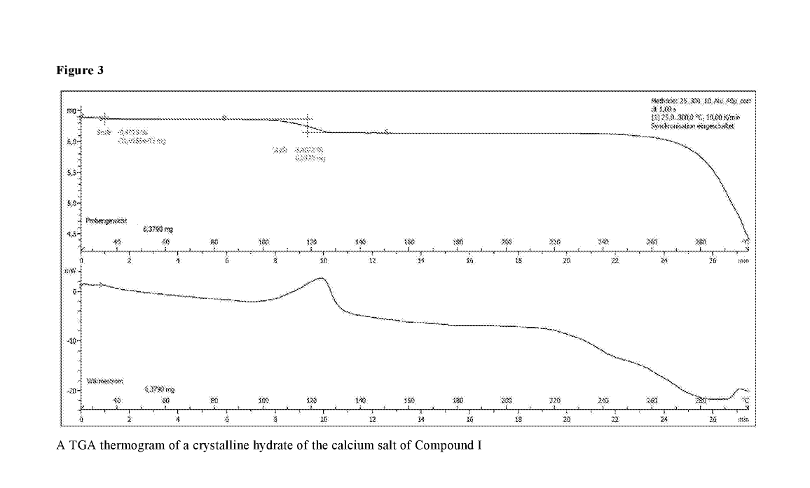

In a further embodiment, the crystalline hydrate of the calcium salt of compound I may be characterized by weight loss of about 3.4% as is obtained in a temperature range of about 110° C. to about 130° C., as measured by TGA (which is in accordance with the theoretical calculated value (3.4%) for one molecule water per Compound I ion). Alternatively, the crystalline hydrate of the calcium salt of compound I may be characterized by TGA thermogram as depicted in FIG. 3.

In a further embodiment, the crystalline hydrate of the calcium salt of compound I of the invention is a mono-hydrate (one molecule water per Compound I ion).

The crystalline hydrate of the calcium salt of compound I of the invention may consist of crystalline hydrate of the calcium salt of compound I. Alternatively, it may also contain small amounts of amorphous hydrate of the calcium salt of compound I. A mixture containing 60 to 99.999% by weight crystalline hydrate of the calcium salt of compound I and 0.001 to 40% by weight amorphous hydrate of the calcium salt of compound I is preferred, more preferably 90 to 99.99% by weight crystalline hydrate of the calcium salt of compound I and 0.01 to 10% amorphous hydrate of the calcium salt of compound I, particularly preferably is the pure form, which is 95 to 99.9% by weight crystalline hydrate of the calcium salt of compound I and 0.1 to 5% amorphous hydrate of the calcium salt of compound I.

In a preferred embodiment the crystalline hydrate of the calcium salt of the present invention is present in solid form at 23° C. This includes that the crystalline hydrate of the calcium salt of the present invention does not liquefy or is not present in a solution.

In a further embodiment crystalline hydrate of the calcium salt can be present in an isolated form. In the context of the invention an isolated form of crystalline hydrate of the calcium salt can be referred to as crystalline hydrate of the calcium salt being in an essentially pure form.

In a preferred embodiment crystalline ethanol solvate of the calcium salt of compound I can preferably have characteristic X-ray powder diffraction peaks at 5.6, 6.0, 12.0 and 16.9 degrees 2θ (±0.2 degrees 2θ).

In a preferred embodiment crystalline ethanol solvate of the calcium salt of compound I can be characterized by one or more further XRPD diffraction peak(s) at 15.4, 18.1 and/or 19.0 degrees 2θ (±0.2 degrees 2θ), preferably at 19.0 degrees 2θ (±0.2 degrees 2θ).

In an alternatively further preferred embodiment of the present invention crystalline ethanol solvate of the calcium salt of compound I can be characterized by the XRPD diffraction peak(s) at degrees 2θ±0.2 degrees 2θ (intensity %): 5.6 (100), 6.0 (10.3), 10.0 (0.2), 10.3 (1.2), 10.7 (1.6), 11.2 (4.7), 12.0 (10.5), 13.9 (0.5), 14.2 (1.2), 14.7 (0.7), 15.4 (0.7), 15.8 (1.4), 16.9 (13.0), 18.1 (8.1) and 19.0 (6.9).

In an alternatively further preferred embodiment of the present invention crystalline ethanol solvate of the calcium salt of compound I can be characterized by the XRPD diffraction peak(s) at degrees 2θ±0.2 degrees 2θ (intensity %): 5.6, 6.0, 10.0, 10.3, 10.7, 11.2, 12.0, 13.9, 14.2, 14.7, 15.4, 15.8, 16.9, 18.1 and 19.0.

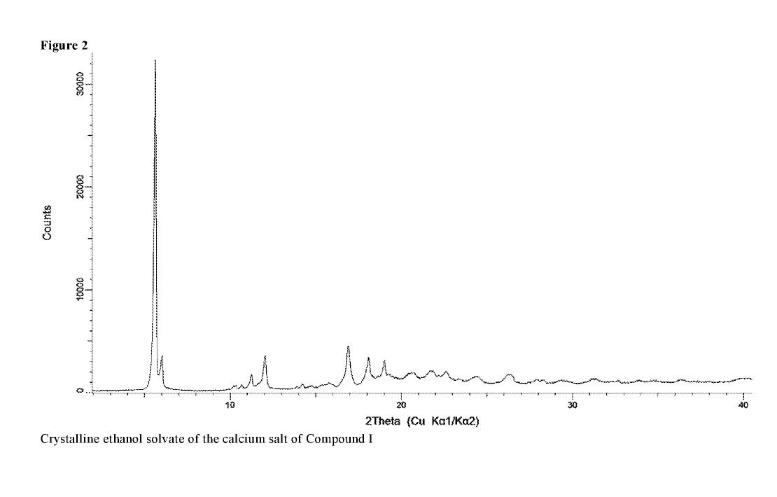

An XRPD diffraction pattern of a preferred embodiment of the crystalline ethanol solvate of the calcium salt of compound I of the present invention is shown in FIG. 2.

In a further embodiment, the crystalline ethanolate of the calcium salt of compound I may be characterized by two weight loss steps of about 7.8% (total), e.g. one step of about 2.8% loss as is obtained in a temperature range of about 40° C. to about 80° C., and another step of about 5.0% loss as is obtained in a temperature range of about 100° C. to about 140° C., as measured by TGA (which is in accordance with the theoretical calculated value (8.3%) for one molecule ethanol per Compound I ion). Alternatively, the crystalline ethanolate of the calcium salt of compound I may be characterized by TGA thermogram as depicted in FIG. 3.

In a further embodiment, the crystalline ethanolate of the calcium salt of compound I of the invention is a mono-ethanolate (mono-ethanol solvate).

The crystalline ethanol solvate of the calcium salt of compound I of the invention may consist of crystalline ethanol solvate of the calcium salt of compound I. Alternatively, it may also contain small amounts of amorphous ethanol solvate of the calcium salt of compound I components. A mixture containing 60 to 99.999% by weight crystalline ethanol solvate of the calcium salt of compound I and 0.001 to 40% by weight amorphous ethanol solvate of the calcium salt of compound I is preferred, more preferably 90 to 99.99% by weight crystalline ethanol solvate of the calcium salt of compound I and 0.01 to 10% amorphous ethanol solvate of the calcium salt of compound I, particularly preferably is the pure form, which is between 95 to 99.9% by weight crystalline ethanol solvate of the calcium salt of compound I and 0.1 to 5% amorphous ethanol solvate of the calcium salt of compound I.

In a preferred embodiment the crystalline ethanol solvate of the calcium salt of the present invention is present in solid form at 23° C. This includes that the crystalline ethanol solvate of the calcium salt of the present invention does not liquefy or is not present in a solution.

In a further embodiment crystalline ethanol solvate of the calcium salt can be present in an isolated form. In the context of the invention an isolated form of crystalline ethanol solvate of the calcium salt can be referred to as crystalline ethanol solvate of the calcium salt being in an essentially pure form.

Depending on which other solid state form it is compared with, the crystalline forms of the Ca salts of Compound I of the present disclosure may have advantageous properties selected from at least one of: chemical or polymorphic purity, flowability, solubility, wettability, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents, adhesive tendencies and advantageous processing and handling characteristics such as compressibility, or bulk density.

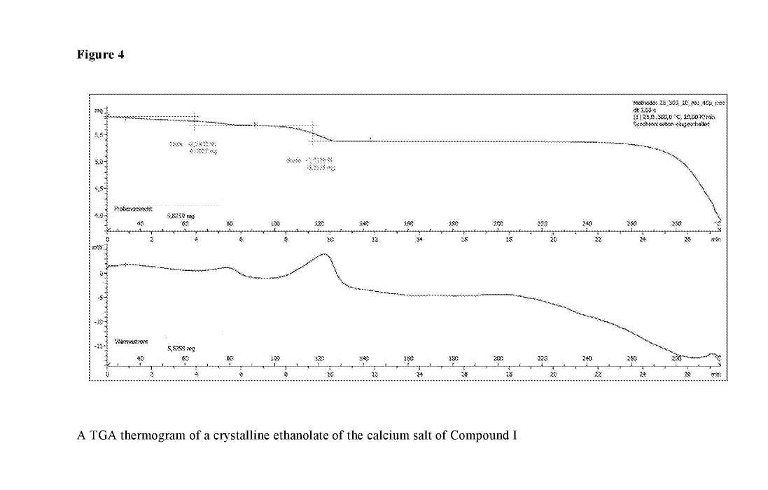

Particularly, while the crystallinity of the Trihydrate (compound 6) and for the dihydrate (compound 7) of the Na salt of Compound I is very poor (according to the XRPD pattern shown in FIGS. 4 and 5 of WO'843), the crystalline forms of the present disclosure are crystalline.

In addition, when compared to other forms of the Na salt of compound I (e.g., Compound 4 (EtOH solvate), Compound 5 (IPA solvate) and Compound 6 (Trihydrate)), which as described in WO'843, are not stable under humid conditions (transform to other forms or lose their crystallinity), the hydrate form of the Ca salt of Compound I of the present disclosure was found to be chemically and polymorphically stable under humidity, i.e., it remains stable after storage in open glass bottles at 25° C./60% RH, 30° C./65% RH, and 40° C./75% RH for 12 weeks. Stability during all processes containing moisture is advantageous regarding e.g. granulation processes (high shear and fluid bed), pellet production (solution/suspension/powder layering on inert cores, direct pelletisation, extrusion/spheronisation processes) or film coating of e.g. tablets, granules or pellets. Also the stability of the morphology of the API as such or in the finished product during storage or transport is advantageous.

A further subject of the present invention is a method of preparing crystalline ethanol solvate of the calcium salt of compound I according to the present disclosure comprising the steps of
(a) providing compound I and calcium hydroxide in a mixture of ethanol and water
(b) heating the mixture from step (a) at boiling temperature

Preferably step (b) is carried out in a reflux system like for example distillation columns or fractionators.

In a preferred embodiment of the invention in step (a) the molar ratio of compound I and calcium hydroxide is from 2.2:1 to 0.8:1, preferably from 2.2:1 to 1:1, more preferably from 2.2:1 to 1.8:1, in particular about 2:1.

In a preferred embodiment of the invention in step (a) the volume ratio of ethanol and water is from 10:1 to 1:10, preferably from 5:1 to 1:5, more preferably from 2:1 to 1:2, in particular about 1.6:1.

In a preferred embodiment of the invention in step (a) 1.0 g of compound I is added to a volume of ethanol/water from 150 mL to 10 mL, preferably from 100 mL to 15 mL, more preferably from 60 mL to 20 mL, in particular about 30 mL.

A further subject of the present invention is a method of preparing crystalline hydrate of the calcium salt of compound I according to the present invention comprising the steps of:
(a) suspending ethanol solvate of the calcium salt in water
(b) stirring the mixture from step (a) and/or storing the mixture from step (a)
(c) isolating crystalline hydrate of the calcium salt of compound I.

The present invention furthermore relates to pharmaceutical compositions comprising crystalline calcium salt of Compound I according to the present invention, wherein the pharmaceutical compositions additionally contain at least one pharmaceutically acceptable excipient.

Pharmaceutically acceptable excipient(s) can for example be fillers, binders, glidants, disintegrants, lubricants, flow regulating agents and release agents. Suitable excipients are for example disclosed in “Lexikon der Hilfsstoffe für Pharmazie, Kosmetik and angrenzende Gebiete”, published by H. P. Fielder, 4^{th} Edition, and “Handbook of Pharmaceutical Excipients”, 3^{rd} Edition, published by A. H. Kibbe, American Pharmaceutical Association, Washington, USA, and Pharmaceutical Press, London.

The term filler generally means substances which serve to form the body of the tablet in the case of tablets with small amounts of active agent (e.g. less than 60% by weight). This means that fillers “dilute” the active agent(s) in order to produce an adequate tablet compression mixture. The normal purpose of fillers therefore is to obtain a suitable tablet size. Examples of preferred fillers are lactose, lactose derivatives, starch, starch derivatives, treated starch, chitin, cellulose and derivatives thereof, calcium phosphate, calcium hydrogen phosphate, sucrose, calcium carbonate, magnesium carbonate, magnesium oxide, maltodextrin, calcium sulphate, dextrates, dextrin and/or dextrose and hydrogenated vegetable oil. Fillers can be present in an amount of 0 to 80% by weight, preferably in an amount of 10 to 60% by weight based on the total weight of the composition.

A binder is generally a substance which is capable of increasing the strength of the resulting dosage form, especially the resulting tablets. Suitable binders are for example polyvinylpyrrolidone, hydroxypropyl cellulose, hydroxypropyl methylcellulose, methylcellulose, hydroxyethyl cellulose, sugars, dextran or corn starch. Binders can be present in an amount of 0 to 30% by weight, preferably in an amount of 2 to 15% by weight based on the total weight of the composition.

Glidants can be used to improve the flowability. Suitable glidants are for example alkaline earth metal salts of fatty acids, like stearic acid. The glidant can be present for example in an amount of 0 to 2% by weight, preferably in an amount of 0.5 to 1.5% by weight based on the total weight of the composition.

Disintegrants are compounds which enhance the ability of the dosage form, preferably the ability of the tablet, to break into smaller fragments when in contact with a liquid, preferably water. Suitable disintegrants are for example croscarmellose sodium, sodium carboxymethyl starch, cross-linked polyvinylpyrrolidone (crospovidone), sodium carboxymethylglycolate and sodium bicarbonate. The disintegrant can be present in an amount of 0 to 20% by weight, preferably in an amount of 1 to 15% by weight based on the total weight of the composition.

A suitable flow regulating agent is for example colloidal silica. The flow regulating agent can be present in an amount of 0 to 8% by weight, preferably in an amount of 0.1 to 3% by weight based on the total weight of the composition.

A suitable release agent is for example talcum. The release agent can be present in an amount of 0 to 5% by weight, preferably in an amount of 0.5 to 3% by weight based on the total weight of the composition.

The pharmaceutical composition can preferably be further processed into an oral dosage form, such as a capsule or tablet.

The oral dosage form, preferably a tablet or a capsule, more preferably a tablet, can preferably be coated, preferably film coated.

In the present invention the following three types of film coatings are possible:
film coatings without affecting the release of the active ingredient,
gastric juice-resistant film coatings,
retard film coatings.

Generally, film coatings can be prepared by using film-forming agents, such as waxes, cellulose derivatives, poly(meth)acrylate, polyvinylpyrrolidone, polyvinyl acetate phthalate, and/or shellac or natural rubbers, such as carrageenan.

It is preferred that the present tablet is coated with a gastric juice-resistant film coating. Alternatively, a capsule comprising a gastric juice-resistant film coating can be used.

The gastric juice-resistant film coating preferably is a film coating being stable in the pH range of about 0.7 to 3.0, which is supposed to be the pH value of human gastric juice found in the stomach. However, in an environment with a pH value of 5 to 9, which is supposed to be present in the (small) intestine of the human body, the gastric juice-resistant film coating preferably dissolves and the drug can be released.

The gastric juice-resistant film coating (often also referred to as enteric coating) can comprise film-forming agents, for example fats, fatty acids, waxes, alginates, shellac, polyvinyl acetate phthalate, cellulose derivatives such as carboxy methyl ethyl cellulose, cellulose acetate succinate, cellulose acetate phthalate, hydroxypropyl methyl cellulose phthalate, hydroxypropyl methyl cellulose acetate succinate, cellulose acetate trimellitate, and meth(acrylic)acid copolymers, such as methyl acrylate-methacrylic acid copolymers, methyl methacrylate-methacrylic acid copolymers and Eudragits (for example Eudragit® L30D, Eudragit® L, Eudragit® S).

The coating is preferably free of active ingredient. It is further preferred that the thickness of the coating is 10 μm to 2 mm, preferably 50 to 500 μm.

The preferred coating may comprise a film-forming agent and one or more of the following: lubricant, surfactant, glidant, pigment and water.

The preferred coating according to an embodiment of the present invention can comprise, along with the film-forming agent, e.g. stearic acid as lubricant for plasticizing and dissolving the polymer, sodium lauryl sulfate as a surfactant for wetting and dispersing, talc as glidant, iron oxide yellow and/or titanium oxide as pigment(s) and optionally purified water.

The present pharmaceutical composition and/or the oral dosage form of the present invention can be prepared by the methods well-known to a person skilled in the art, such as dry and wet granulation and direct compression.

In a preferred embodiment, the pharmaceutical composition and/or the oral dosage form can be administered preferably twice a day.

In another embodiment, the present disclosure encompasses the use of the above described solid state forms of the Ca salt of Compound I for the preparation of pharmaceutical compositions.

The present invention further relates to the crystalline calcium forms of compound I, or to any of the pharmaceutical compositions according to the present invention for use in the treatment of pain, preferably for use in the treatment of inflammatory pain.

The present disclosure also provides methods of treating inflammatory pain, comprising administering a therapeutically effective amount of a solid state form of the Ca salt of Compound I of the present disclosure, or at least one of the above described pharmaceutical compositions, to a subject suffering from pain, or otherwise in need of the treatment.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### EXPERIMENTAL PART

### Analytical Methods

X-ray Powder Diffraction (XRPD)

The samples were measured on a D8 Advance powder X-ray diffractometer (Bruker AXS, Karlsruhe, Germany) in a rotating PMMA sample holder (diameter: 25 mm; depth: 1 mm) in reflection mode (Bragg-Brentano geometry). Conditions of the measurements are summarized in the table below. Raw data were analyzed with the program EVA (Bruker AXS, Karlsruhe, Germany). Background subtraction and Kα₂ stripping were not performed for the depicted diffractograms. Peak intensities were determined after background subtraction.

Conditions for powder diffraction measurements were as follows:

| | |
|---|---|
| Radiation | Cu Kα₁/α₂ |
| Source | 34 kV/40 mA |
| Detector | Vantec-1 (electronic window: 3°) |
| Kβ filter | Ni (diffracted beam) |
| measuring circle diameter | 435 mm |
| detector window slit | 12 mm |
| anti-scatter slit (diffracted beam) | 8 mm |
| divergence slit | v6.00 (variable) |
| Soller slit (incident/diffracted beam) | 2.5° |
| 2θ range | 2° ≤ 2θ ≤ 55° |
| step size | 0.016 |
| step time | 0.2 s |

### Thermogravimetric Analysis (“TGA”)

| | |
|---|---|
| Apparatus: | Mettler Toledo TGA/DSC1 (Mettler-Toledo GmbH, |
| | Gießen, Germany) |
| Aluminium crucible: | 40 μL (open) |
| Temperature range: | 25° C. to 300° C. |
| Heating rate: | 10° C./min |

### Differential Scanning Calorimetry (DSC)

The samples were placed in sealed aluminum crucibles (40 μL) with perforated lids (one hole in the centre, made by puncturing with a cannula of 0.6 mm diameter). The measured DSC curves are displayed as a function of the program temperature (proportional to the measurement time). Characteristic temperature values of DSC signals (onset/endset temperature, peak maximum) are determined from the sample temperature, which may deviate from the program temperature. Signals with positive area correspond to endothermic events.
Apparatus: Mettler-Toledo DSC 822E coupled with a Mettler-Toledo Gas-Flow-Controller TS0800GC1 (Mettler-Toledo GmbH, Gießen, Germany)
Aluminium crucible: 40 μL (with perforated lid)
Temperature range: 30° C. to 300° C.
Heating rate: 10° C./ min
Nitrogen flow: 50 mL/min
Software: STARe Version 12.00a
Interpretation: Endothermic mode

### Determination of Chemical Purity (HPLC-MS)

Measurements were performed by using the following specifications:

| Instrument: | Agilent 1200 coupled with Bruker Ion trap HCT | | |
|---|---|---|---|
| Wavelength: | 278 nm | | |
| Column: | Phenomenex Kinetex C18 150 * 4.6 mm 2.6 μm | | |
| Column temp.: | 40.0° C. | | |
| Flow [mL/min]: | 1.0 | | |
| Injection volume: | 2 μL | | |
| Solvent A: | Acetonitrile | | |
| Solvent B: | Water + 0.2% formic acid + 0.1% heptafluorobutyric | | |
| | acid | | |
| | Time (min) | A | B |
| Gradient | 0.00 | 45% | 55% |
| | 8.00 | 85% | 15% |
| | 10.00 | 85% | 15% |
| | 10.10 | 45% | 55% |

### Determination of Enantiomeric Purity (HPLC-MS)

Enantiomeric purity is defined as [(main enantiomer)/(sum of both enantiomers)]. Measurements were performed by using the following specifications:
Instrument: Agilent 1200 coupled with Bruker Ion trap HCT
Wavelength: 278 nm
Column: Phenomenex Lux Cellulose 4 250*4.6 mm 3 μm
Flow [mL/min]: 1.2
Injection volume: 2
Solvent: Acetonitrile+0.2% formic acid
Gradient isocratic

### EXAMPLES

### Reference Example

Compound I was purchased as an amorphous, slightly yellow solid. The chemical purity determined by HPLC was 94.5%. The enantiomeric purity was 97.3% (main enantiomer/sum of both enantiomers). The material contained approx. 2.5% of ethyl acetate (GC-MS) as residual solvent. Alternatively, Compound I may be prepared according to the process described in WO2012010843.

### Example 1

### Preparation of Crystalline Ethanol Solvate of the Calcium Salt of Compound I

Compound I (15.00 g; 29.6 mmol) was added at room temperature to a suspension of calcium hydroxide (1.09 g; 14.8 mmol) in a mixture of ethanol (275 mL) and deionized water (175 mL). The suspension was heated at boiling temperature under reflux and stirring. A voluminous solid precipitated initially which dissolved at boiling temperature. The mixture was refluxed for 20 min at boiling temperature, then it was filtered hot through a folded filter. The clear filtrate was cooled to room temperature under ambient conditions. Crystallization of the product started immediately. After storage at room temperature overnight the crystallized solid was isolated by vacuum filtration and dried at 5 mbar/room temperature overnight. A colorless solid was obtained. The yield was 10.89 g.

The chemical purity of the product determined by HPLC was 99.2%, the enantiomeric purity was 99.9%.

Crystalline ethanol solvate of the calcium salt of compound I can be characterized by the XRPD diffraction peak(s) at degrees 2θ±0.2 degrees 2θ (intensity %): 5.6 (100), 6.0 (10.3), 10.0 (0.2), 10.3 (1.2), 10.7 (1.6), 11.2 (4.7), 12.0 (10.5), 13.9 (0.5), 14.2 (1.2), 14.7 (0.7), 15.4 (0.7), 15.8 (1.4), 16.9 (13.0), 18.1 (8.1) and 19.0 (6.9).

The X-ray powder diffractogram is shown in FIG. 2.

The DSC thermogram gave two endothermic signals, which are both due to solvent loss.

### Example 2

### Preparation of Crystalline Hydrate of the Calcium Salt of Compound I

The ethanol solvate of the calcium salt of Compound I (3.0 g) was stirred as a suspension in deionized water (36 mL) at room temperature for 5 h. The suspension was then stored overnight at room temperature without stirring. The solid was isolated by vacuum filtration, washed with deionized water (25 mL) and dried at 5 mbar/room temperature for 3.5 h. A colorless solid was obtained. The yield was 2.62 g.

The chemical purity of the product determined by HPLC was 99.2%, the enantiomeric purity was 100.0%. The content of ethanol (GC-MS) was approx. 760 ppm.

Crystalline hydrate of the calcium salt of compound I can be characterized by the XRPD diffraction peak(s) at degrees 2θ±0.2 degrees 2θ (intensity %): 5.6 (100), 6.0 (6.1), 9.9 (1.6), 10.5 (0.8), 11.2 (0.2), 11.9 (9.5), 12.9 (0.3), 13.3 (0.7), 14.5 (0.4), 15.0 (2.2), 15.4 (2.0), 16.1 (3.4), 16.7 (9.2), 16.8 (13.1), 17.3 (0.5), 17.9 (9.3), 18.2 (2.1), 18.6 (0.5), 19.3 (4.2), 19.5 (4.1), 19.9 (2.1), 20.3 (0.8), 21.2 (16.4), 21.5 (6.5), 22.2 (2.1), 22.5 (2.3) and 23.3 (8.3).

The X-ray powder diffractogram is shown in FIG. 1.

The DSC thermogram gave two endothermic signals, which are both due to solvent loss.

### Example 3

### Preparation of Crystalline Ethanol Solvate of the Calcium Salt of Compound I

Compound I (60.00 g; 118 mmol) was added to a suspension of calcium hydroxide (4.38 g; 59 mmol) in a mixture of ethanol (1500 mL) and deionized water (700 mL). The mixture was heated at reflux temperature for 20 min and then filtered hot through a folded filter. The residue in the filter was washed with hot ethanol (50 mL). The combined filtrates were cooled to room temperature under mechanical stirring. Crystallization started immediately. The mixture was stirred overnight at room temperature, the solid was then isolated by vacuum filtration, washed with ethanol (200 mL), and dried under vacuum (4 mbar) at room temperature overnight to provide the crystalline ethanol solvate of the Ca salt of Compound I (as was confirmed by XRPD).

1. Crystalline calcium salt of (S)-2-(diphenylacetyl)-1,2,3,4-tetrahydro-6-methoxy-5-(phenylmethoxy)-3-isoquinoline carboxylic acid.

2. Crystalline hydrate of the calcium salt of (S)-2-(diphenylacetyl)-1,2,3,4-tetrahydro-6-methoxy-5-(phenylmethoxy)-3-isoquinoline carboxylic acid according to claim 1, having characteristic X-ray powder diffraction peaks at 5.6, 6.0, 11.9 and 16.8 degrees 2θ (±0.2 degrees 2θ).

3. Crystalline hydrate of the calcium salt of (S)-2-(diphenylacetyl)-1,2,3,4-tetrahydro-6-methoxy-5-(phenylmethoxy)-3-isoquinoline carboxylic acid according to claim 2, further characterized by one or more XRPD diffraction peak(s) at 9.9, 15.0, 15.4 and/or 16.1 degrees 2θ (±0.2 degrees 2θ).

4. Crystalline ethanol solvate of the calcium salt of (S)-2-(diphenylacetyl)-1,2,3,4-tetrahydro-6-methoxy-5-(phenylmethoxy)-3-isoquinoline carboxylic acid according to claim 1, having characteristic X-ray powder diffraction peaks at 5.6, 6.0, 12.0 and 16.9 degrees 2θ (±0.2 degrees 2θ).

5. Crystalline ethanol solvate of the calcium salt of (S)-2-(diphenylacetyl)-1,2,3,4-tetrahydro-6-methoxy-5-(phenylmethoxy)-3-isoquinoline carboxylic acid according to claim 4, further characterized by one or more XRPD diffraction peak(s) at 15.4, 18.1 and/or 19.0 degrees 2θ (±0.2 degrees 2θ).

6. Method for the preparing crystalline ethanol solvate of the calcium salt of (S)-2-(diphenylacetyl)-1,2,3,4-tetrahydro-6-methoxy-5-(phenylmethoxy)-3-isoquinoline carboxylic acid according to claim 1 comprising the steps of:
(a) providing (S)-2-(diphenylacetyl)-1,2,3,4-tetrahydro-6-methoxy-5-(phenylmethoxy)-3-isoquinoline carboxylic acid and calcium hydroxide in a mixture of ethanol and water; and
(b) heating the mixture from step (a) at boiling temperature.

7. Method according to claim 6, wherein in step (a) the molar ratio of (S)-2-(diphenylacetyl)-1,2,3,4-tetrahydro-6-methoxy-5-(phenylmethoxy)-3-isoquinoline carboxylic acid and calcium hydroxide is from 2.2:1 to 0.8:1.

8. Method according to claim 6, wherein in step (a) the volume ratio of ethanol and water is from 10:1 to 1:10.

9. Method of preparing crystalline hydrate of the calcium salt of (S)-2-(diphenylacetyl)-1,2,3,4-tetrahydro-6-methoxy-5-(phenylmethoxy)-3-isoquinoline carboxylic acid according to claim 1 comprising the steps of:
(a) suspending ethanol solvate of the calcium salt in water;
(b) stirring the mixture from step (a) and/or storing the mixture from step (a); and
(c) isolating crystalline hydrate of the calcium salt of (S)-2-(diphenylacetyl)-1,2,3,4-tetrahydro-6-methoxy-5-(phenylmethoxy)-3-isoquinoline carboxylic acid.

10. A pharmaceutical composition comprising crystalline calcium salt of (S)-2-(diphenylacetyl)-1,2,3,4-tetrahydro-6-methoxy-5-(phenylmethoxy)-3-isoquinoline carboxylic acid according to claim 1 and further at least one pharmaceutically acceptable excipient.

11. Crystalline calcium salt of (S)-2-(diphenylacetyl)-1,2,3,4-tetrahydro-6-methoxy-5-(phenylmethoxy)-3-isoquinoline carboxylic acid according to claim 1 for use in the treatment of pain.

12. Method for treating and/or preventing pain comprising administering to a subject in need thereof a therapeutically effective amount of the crystalline calcium salt of (S)-2-(diphenylacetyl)-1,2,3,4-tetrahydro-6-methoxy-5-(phenylmethoxy)-3-isoquinoline carboxylic acid according to claim 1.

13. Use of the crystalline form according to claim 1 in the manufacture of pharmaceutical compositions and/or formulations.

14. Use of the crystalline form according to claim 1 for the manufacture of a medicament for treating pain.

15. Method for treating and/or preventing pain comprising administering to a subject in need thereof a therapeutically effective amount of the pharmaceutical composition according to claim 10.

16. Use of the pharmaceutical composition according to claim 10 for the manufacture of a medicament for treating pain.
